(19) United States
(12) Patent Application Publication
(10) Pub. No.: US 2010/0227831 A1
(43) Pub. Date: Sep. 9, 2010

(54) COMPOSITIONS AND METHODS FOR CANCER TREATMENT

(76) Inventor: Jayanta Saha, Apex, NC (US)

Correspondence Address: WILLIAMS MULLEN, 222 CENTRAL PARK AVENUE, SUITE 1700, VIRGINIA BEACH, VA, 23462, US

(21) Appl. No.: 12/782,774

(22) Filed: May 19, 2010

Related U.S. Application Data
(63) Continuation of application No. 11/960,031, filed on Dec. 19, 2007.
(60) Provisional application No. 60/883,563, filed on Jan. 5, 2007.

(51) Int. Cl. A61K 31/704; C07H 15/252; C07F 9/535; A61K 31/675; C07H 19/06; A61K 31/7068; A61P 35/00

(52) U.S. Cl. 514/34; 536/6.4; 546/23; 514/81; 544/243; 514/86; 536/26.8; 514/49

(57) ABSTRACT

A novel composition of a phosphonoformic acid partial ester chemically linked to an anticancer compound of the general formula:

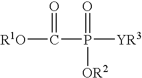

wherein R¹ is selected from the group consisting of methyl, alkyl, cholesteryl, aryl and aralkyl, R² is selected from the group consisting of hydrogen, methyl, alkyl, and a water-soluble cation, Y is selected from the group consisting of oxygen, sulfur, carbon and nitrogen, and R³ is a cytotoxic agent. Methods for making and administering these compositions for treatment of cancer are also disclosed.

This application is a continuation patent application based upon and claims the benefit of U.S. application Ser. No. 11/960,031 filed Dec. 19, 2007, and U.S. Provisional Application No. 60/883,563, filed Jan. 5, 2007, the disclosures of each of which are incorporated herein in their entireties.

### FIELD OF THE INVENTION

The present invention relates generally to the fields of reducing drug toxicity and enhancing drug efficacy during chemotherapy. More particularly, the present invention relates to novel treatment methods, compositions and kits comprising a phosphonoformic acid partial ester chemically linked to an anticancer compound. These novel compounds of the present invention have been found to lead to a pronounced decrease in toxicity, enhanced synergy, and increased effectiveness against drug-resistant cancers over and above the actions of the individual components alone or in combination.

### BACKGROUND OF THE INVENTION

The term “cancer” generally refers to any of a group of more than 100 diseases caused by the uncontrolled growth of abnormal cells. Cancer can take the form of solid tumors and lymphomas, and non-solid cancers such as leukemia. Unlike normal cells, which reproduce until maturation and then only as necessary to replace wounded cells, cancer cells can grow and divide endlessly, crowding out nearby cells and eventually spreading to other parts of the body.

Because cancer cells generally divide more frequently than normal cells, the majority of drug-mediated chemotherapies rely on cytotoxic agents that selectively poison dividing cells. For the past several decades, many cytotoxic agents have been developed that target different aspects cellular growth, such as the inhibition of cell cycle proteins and processes, inhibition of signal transduction proteins and pathways, inhibition of microtubule formation, inhibition of DNA replication and the like. Several of these cytotoxic agents have attained a certain degree of success, including, but not limited to, anthracyclines, such as daunomycin, adriamycin (doxorubicin), epirubicin, and idarubicin, quinoline-based alkaloids such as camptothecin, aminocamptothecin, irinotecan, topotecan, and DX-8951f, imatinib mesylate (Gleevec®), methotrexate, mithramycin, cytosine arabinoside, 6-azauridine, paclitaxel and the like. While these cytotoxic agents have proven somewhat successful in treating many types of cancer, each exhibits a comparatively high degree of toxicity to the patient. This toxicity presents a challenge to the practitioner to deliver an adequate dosage of cytotoxic agent(s) to effectively eliminate the cancer but also keep the toxicity of the cytotoxic agent low enough to not harm the mammal being treated. This delicate balance, if not properly monitored, can result in either the cancer not being completely eliminated or death to the patient.

Therefore, there exists a need to identify new cytotoxic agents that are effective in killing cancer cells yet protect normal host tissues from the undesired toxicity of the cytotoxic agent.

### SUMMARY OF THE INVENTION

The present invention seeks to overcome these and other drawbacks inherent in the prior art by providing new treatment methods, compositions, and kits for reducing the toxicity of currently utilized chemotherapy drugs by chemically linking a phosphonoformate ester with the desired chemotherapy drug.

One aspect of the invention relates to a chemotherapeutic agent comprising the structure

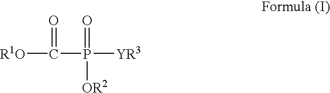

wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is a cytotoxic agent, or a pharmaceutically acceptable salt, ester, or other physiologically functional derivative thereof.

In one embodiment, the invention relates to a chemotherapeutic agent having the structure

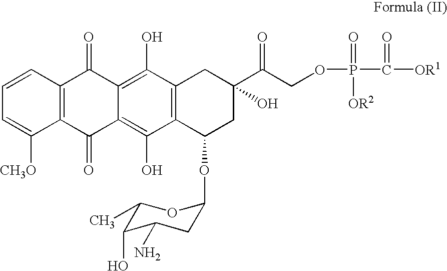

wherein R¹ is selected from the group consisting of methyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In another embodiment, the invention relates to a chemotherapeutic agent having the structure

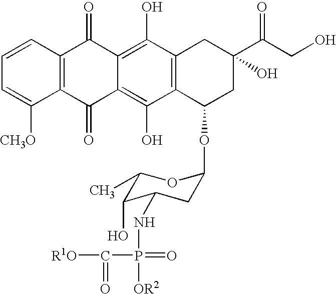

### Formula (III)

wherein R¹ is selected from the group consisting of methyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In another embodiment, the invention relates to a chemotherapeutic agent having the structure

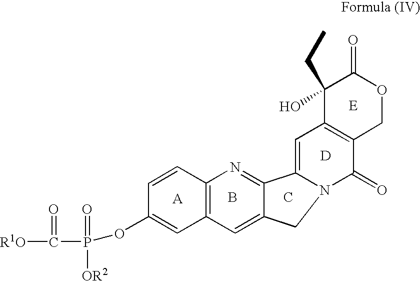

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In yet another embodiment, the invention relates to a chemotherapeutic agent having the structure

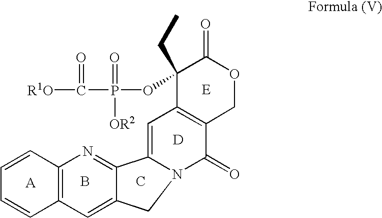

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In another embodiment, the invention relates to a chemotherapeutic agent having the structure

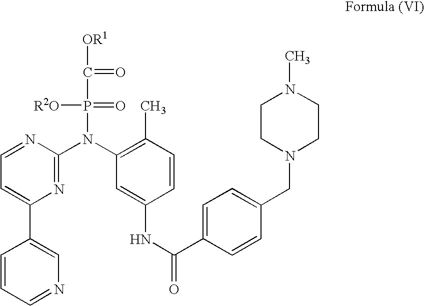

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In another embodiment, the invention relates to a chemotherapeutic agent having the structure

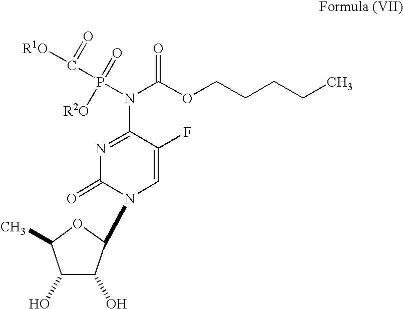

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

Another aspect of the present invention relates to a method of treating or preventing a cancerous condition in a patient comprising administering to the patient an effective amount of a compound of the formula

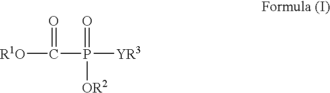

wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is a cytotoxic agent, or a pharmaceutically acceptable salt, ester, or other physiologically functional derivative thereof, whereby the cancerous condition is treated or prevented in the patient. In one embodiment, R¹ is methyl or ethyl, Y is oxygen or nitrogen, R² is ammonium, and R³ is adriamycin, camptothecin, imatinib mesylate, or capecitabine.

Yet another embodiment of the present invention relates to a method of inhibiting the cell growth in a patient suffering from a cancerous condition comprising the steps of administering to the patient an effective amount of a chemotherapeutic agent of the present invention such that the growth of the cells of the cancerous condition is inhibited in the patient.

In another embodiment, the present invention relates to a method of treating a patient suffering from a cancerous condition comprising the steps of administering to the patient an effective amount of a chemotherapeutic agent of the present invention whereby the leukemia is treated in the patient.

In another embodiment, the present invention relates to a method of inhibiting or preventing the growth of a cancerous condition in a patient comprising the steps of administering to the patient effective amount of a chemotherapeutic agent of the present invention whereby the growth of the leukemia is inhibited or prevented in the patient.

In yet another embodiment, the methods further comprise the addition of at least one additional chemotherapeutic drug.

In another embodiment, the cancerous condition is selected from the group consisting of breast cancer, ovarian cancer, transitional cell bladder cancer, bronchogenic lung cancer, thryroid cancer, gastric cancer, soft tissue sarcomas, osteogenic carcinomas, neuroblastomas, Adjuvant Stage H1 Dukes' C colon cancer, Wilms' tumor, malignant lymphoma, Hodgkin's lymphoma, Non-Hodgkin's lymphoma, acute myelogenous leukemia (AML), acute lymphoblastic leukemia (ALL), Kaposi's sarcoma, small cell lung cancer, colorectal cancer, and chronic myeloid leukemia (CML). In one embodiment, the cancerous condition is acute myelogenous leukemia, acute lymphoblastic leukemia or chronic myeloid leukemia.

Another aspect of the present invention relates to a pharmaceutical composition comprising an effective amount of the formula

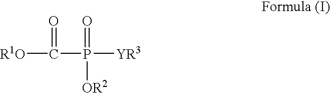

wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is a cytotoxic agent, or a pharmaceutically acceptable salt, ester, or other physiologically functional derivative thereof; and a pharmaceutically acceptable excipient. In one embodiment, the pharmaceutical composition is suitable for injection, intrathecal administration, parenteral administration and/or oral administration. In another embodiment, the pharmaceutical composition is in unit dosage form.

Another aspect of the invention relates to a kit comprising a therapeutically effective amount of the chemotherapeutic agent of the present invention, a pharmaceutically acceptable excipient, and instructions describing its use for the treatment of a cancerous condition. In one embodiment, the cancerous condition is selected from the group consisting of breast cancer, Adjuvant Stage H1 Dukes' C colon cancer, breast cancer, acute myelogenous leukemia (AML), acute lymphoblastic leukemia (ALL) and chronic myelogenous leukemia (CML).

These chemotherapeutic agents of the present invention have utility in medical therapy, in particular, for treating a cancerous condition in a patient. Various other aspects, features and embodiments of the invention will be more fully apparent from the ensuing discussion and appended claims.

### DETAILED DESCRIPTION

The vast majority of currently used cytotoxic agents, including anthracyclines, such as daunomycin, adriamycin (doxorubicin), epirubicin, and idarubicin, quinoline-based alkaloids such as camptothecin, aminocamptothecin, irinotecan, topotecan and DX-8951f, imatinib mesylate (Gleevec®), capecitabine (Xeloda®) methotrexate, mithramycin, cytosine arabinoside, 6-azauridine, paclitaxel and the like have been shown to be effective against many forms of cancer. However, as has been well established, these compounds, while highly efficacious for the treatment of various types of cancer, have accompanying side effects, including high rates of toxicity (e.g. cardiotoxicity). The use of the chemotherapeutic agents according to one embodiment of the present invention may significantly decrease drug-related toxicity, enhance synergy between other chemotherapeutic drugs and provide increased efficacy against drug-resistant cancers.

One embodiment of the present invention therefore provides, inter alia, novel chemotherapeutic agents, methods of using said chemotherapeutic agents for reducing, treating or preventing a cancerous condition in a patient and pharmaceutical compositions comprising an effective amount of one or more chemotherapeutic agents and pharmaceutically acceptable excipient. The uses of the chemotherapeutic agents of the present invention for combating cancer, and in combination pharmaceutical compositions, are discussed below.

The present invention relates to a chemotherapeutic agent having the structure

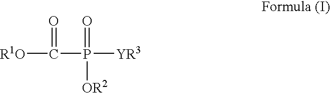

wherein R¹ is selected from the group consisting of methyl, alkyl, cholesteryl, aryl and aralkyl, R² is selected from the group consisting of hydrogen, methyl, alkyl, and a water-soluble cation, Y is selected from the group consisting of oxygen, sulfur, carbon and nitrogen and R³ is a cytotoxic agent, or a pharmaceutically acceptable salt, ester, or other physiologically functional derivative thereof.

The phosphonoformic acid partial ester portion of the compound of the present invention contains as the carboxyl ester group R¹ which may comprise a methyl, alkyl, cholesteryl, aryl or aralkyl group. In one embodiment, the R¹ group comprises an alkyl group having from one to five carbon atoms; in another embodiment one to three carbon atoms. In another embodiment, the R¹ group is either a methyl or ethyl. In addition, the phosphinic acid portion designated R² above may be hydrogen, methyl, alkyl or in the form of a water soluble cation such as sodium, ammonium, or quaternary ammonium, or amine. In one embodiment, R² is ammonium. Moreover, the portion designated Y can be oxygen, nitrogen, carbon or sulfur. In one embodiment, Y is oxygen. However, there are some species of cytotoxic agents which will bind the phosphonoformic acid ester at a nitrogen, whereby Y will be a nitrogen. Lastly, the R³ portion of the phosphonoformic acid partial ester comprises a cytotoxic agent.

The bond linking the phosphonoformic acid partial ester of the present invention to the cytotoxic agent (R³) may include, but is not limited to, covalent bond or non-covalent, polar covalent bond, ionic bond, coordinate covalent bond, banana bond, permanent dipole to permanent dipole bond, hydrogen bond, instantaneous dipole to induced dipole (van der Waals forces) bond, cation-pi interaction and the like. Further, the bond may also be hydrolyzable or non-hydrolyzable. Notably the components may be indirectly covalently bonded or indirectly covalently bonded to one another through an intervening moiety or component, such as a bridge, spacer or linker or the like, e.g., a sugar moiety, glycerin moiety or peptide moiety. Further, the spacer, bridge, linker or the like may also contain sites that are cleavable by enzymes.

As used herein, the term “covalent bond” refers to the type of bonding in which the electronegativity difference between the bonded atoms is small or non-existent. This term also includes the many variations of covalent bonds described below, including polar covalent bond, coordinate covalent bond and the like. The term “polar covalent bond” refers to the type of bonding that is intermediate between a covalent bond and ionic bond. The term “ionic bond” refers to those chemical linkages between two atoms caused by electrostatic forces between oppositely-charged ions in an ionic compound. Common examples of these types of bonds include those between the sodium and chloride ions in salt (NaCl). Generally, ionic charges in an ionic bond are between −3e to +7e. The term “coordinate covalent bond,” also known as “dative bonding,” refers to the type of covalent bond where electrons originate solely from one of the atoms, the electron-pair donor, or Lewis base but are approximately equally shared in the formation of a covalent bond. This type of bonding commonly occurs in nitrons and ammonia borane. The term “banana bond” refers to the type of bonding where the bond bends, often due to the presence of an influencing atom in the middle of another covalent bond. These bonds are likely to be more susceptible to reactions than ordinary bonds. The term “permanent dipole to permanent dipole” refers to a large electronegativity difference between two strongly bonded atoms within a molecule that causes a dipole (i.e. a pair of permanent partial charges) to form. Dipoles either attract or repel each other. The term “hydrogen bond” refers to those bonds where the hydrogen proton comes closer to being shared between target and donor atoms, in a three-center two-electron bond. The term “instantaneous dipole to induced dipole (van der Waals forces) refers to the weakest, but most prolific, bonding formation where an otherwise neutral atom is slightly imbalanced, and thus capable of momentarily being able to attract or repel electrons within a neighboring atom. The term “cation-pi” refers to those interactions that occur between the localized negative charge of [symbol] orbital electrons, located above and below the plane of an aromatic ring, and a positive charge.

As used herein, the terms “cytotoxic agent,” “anticancer drug,” “chemotherapeutic agent,” “chemotherapeutic compound,” and/or “chemotherapeutic drug” relate to any anticancer drug which acts by killing, inhibiting or preventing the division (e.g. replication) of cells and exhibits an undesirable level of toxicity when administered to a patient. The term “undesirable level” as used herein is defined as those side effects that are unwanted, whether they are life threatening or merely inconvenient or uncomfortable to the patient. Examples of cytotoxic agents which are within the scope of the present invention include, but are not limited to, (1) alkylating agents, such as nitrogen mustards (e.g. Chlorambucil, Chlormethine, Cyclosphosphamide, Ilosfamide, and Melphalan), nitrosoureases (e.g. Carmustine, Fotemustine, Lomustine, and Streptozotocin), platinums (e.g. Carboplatin, Oxaliplatin, and BBR3464) and agents such as Busulfan, Dacarbazine, Mechlorethamine, Procarbazine, Temozolomode, ThioTEPA and Uramustine; (2) antimetabolites, such as Folic acid (e.g. Methotrexate, Pemetrexed, Raltitrexed), Purines (e.g. Cladribine, Clofarabine, Fludarabine, Mercaptopurine, Pentostatin and Tioguanine) and Pyrimidines (e.g. Capecitabine) as well as Cytarabine, Fluorouracil and Gemcitabine; (3) plant alkyloids and terpenoids such as Taxane (e.g. Docetaxel and Paclitaxel), Vincas (e.g. Vinblastine, Vincristine, Vindesine and Vinorelbine) and Podophyllotoxins (e.g. etoposide and teniposide); (4) cytotoxic/antitumor antibiotics, such as anthracyclines (e.g. Adriamycin, Daunorubicin, Doxorubicin, Epirubicin, Idarubicin, Mitoxantrone and Valrubicin) as well as Bleomycin, Hydroxyurea and Mitomycin; (5) Topoisomerase inhibitors, such as Topotecan, Camptothecin, Aminocamptothecin, Irinotecan (CPT-11); (6) Kinase inhibitors, such as Dasatinib, Erlotinib, Gefitinib, Lapatinib, Nilotinib, imatinib (Gleevec®), Sorafenib, Sunitinib, and Vandetanib; (7) retinoids, such as Alitretinoin, Tretinoin (all trans retinoic acid), Bexarotene; (8) enzyme and enzyme inhibitors such as Asparaginase and Hydroxycarbamide; (9) proteosome inhibitors such as Bortezomib; and (10) other cytotoxic agents such as 3-aminopyridine-2-carboxaldehyde thiosemicarbazone, Avicine, Actinomycin, Altretamine, Amsacrine, Anagrelide, Anthracycline, Arsenic trioxide, 5-azacytidine, Azathioprine, Bis-(2-chloroethyl)ethylamine, Bryostatin, Cladribine, Clofarabine, Dacarbazine, Dasatinib, Decitabine, Denileukin diftitix, Discodermolide, Epothilone, Estramustine, Exisulind, Floxuridine, Folinic acid, Lenalidomide, Masoprocol, Mechlorethamine, Melphalan, Mitotane, Mesna, Nelarabine, PAC-1, Pegaspargase, Procarbazine, Rasburicase, Temozolomide, Uracil Mustard, Valrubicin and Zosuquidar trihydrochloride, as well as other chemotherapeutic drugs known to those skilled in the art of oncology and any pharmaceutically acceptable salt, ester, or other physiologically functional derivative thereof. In one embodiment, the cytotoxic agent is selected from the group consisting of adriamycin, camptothecin, capecitabine, iminitab and any pharmaceutically acceptable salt, ester, or other physiologically functional derivative thereof.

As used herein, in reference to the present invention, the term “alkyl” is intended to be broadly construed and encompassing: (i) allyl groups of straight-chain as well as branched chain character; (ii) unsubstituted as well as substituted alkyl groups, wherein the substituents of substituted alkyl groups may include any sterically acceptable substituents which are compatible with such alkyl groups and which do not preclude the efficacy of the formula (I) of the present invention for its intended utility (examples of substituents for substituted alkyl groups include halo, amino, amido, C1-C4 alkyl, C₁-C₄ alkoxy, nitro, hydroxyl, etc.); (iii) saturated alkyl groups as well as unsaturated alkyl groups, the latter including groups such as alkenyl-substituted alkyl groups (e.g. allyl, methallyl, propallyl, butenylmethyl, etc.), alkynyl-substituted alkyl groups, and any other alkyl groups containing sterically acceptable unsaturation which is compatible with such alkyl groups and which does not preclude the efficacy of the formula (I) of the present invention for its intended utility; and (iv) alkyl groups including linking or bridge moieties, e.g. heteroatoms such as nitrogen, oxygen, sulfur, etc.

As used herein, in reference to the present invention, the term “hydrocarbyl” is intended to encompass a group containing only carbon and hydrogen atoms, which may contain double or triple bonds and which may be cyclic or aromatic in nature.

As used herein, the term “aryl” also is intended to be broadly construed as referring to carboxylic as well as heterocyclic aromatic groups and encompassing unsubstituted as well as substituted aryl groups, wherein the substituents of substituted aryl groups may include any sterically acceptable substituents which are compatible with such aryl groups and which do not preclude the efficacy of the formula (I) of the present invention for its intended utility (examples of substituents for substituted aryl groups include halo, amino, amido, C₁-C₄ alkyl, C₁-C₄ alkoxy, nitro, hydroxyl, hydroxyalkyl containing a C₁-C₄ alkyl moiety, etc.).

As used herein, the term “aralkyl” is intended to be construed broadly as referring to a radical in which an aryl group is substituted for an alkyl H atom, e.g., those derived from an arylated alkyl.

In one embodiment, the present invention relates to a chemotherapeutic agent having the structure

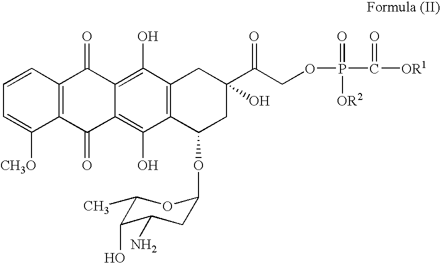

wherein R¹ is selected from the group consisting of methyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In another embodiment, the present invention relates to a chemotherapeutic agent having the structure

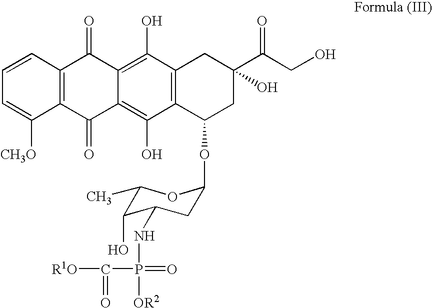

wherein R¹ is selected from the group consisting of methyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In yet another embodiment, the present invention relates to a chemotherapeutic agent having the structure

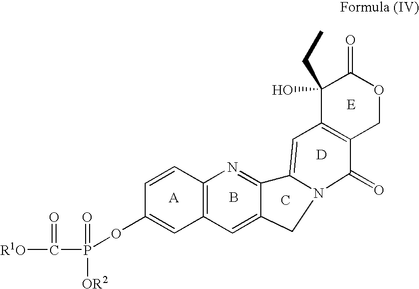

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In another embodiment, the present invention relates to a chemotherapeutic agent having the structure

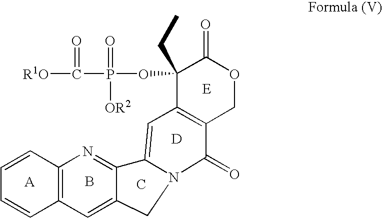

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

In another embodiment, the present invention relates to a chemotherapeutic agent having the structure

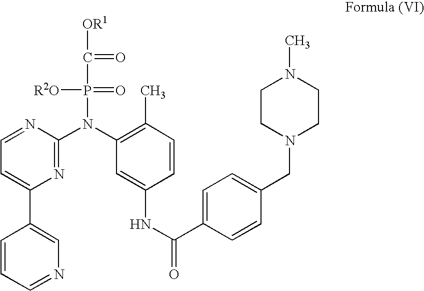

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In another embodiment, R¹ is methyl and R² is ammonium. In another embodiment, R¹ is ethyl and R² is ammonium.

In yet another embodiment, the present invention relates to a chemotherapeutic agent having the structure

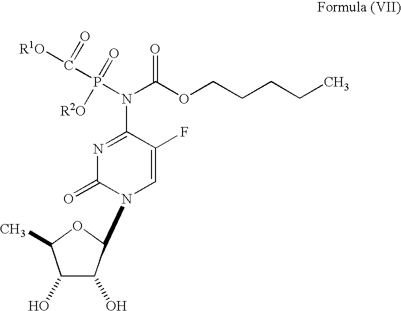

wherein R¹ is selected from the group consisting of methyl, alkyl, cholesteryl, aryl and aralkyl and R² is selected from the group consisting of hydrogen, methyl, alkyl and water soluble cation, or a pharmaceutically acceptable salt, ester, or other physiologically functional derivative thereof. In one embodiment, R¹ is methyl and R² is ammonium. In another embodiment, R¹ is ethyl and R² is ammonium.

Another aspect of the present invention relates to a method of treating or preventing a cancerous condition in a patient, comprising administering to the patient a therapeutically effective amount of a compound of the formula

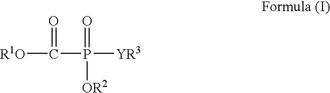

wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is a cytotoxic agent, or a pharmaceutically acceptable salt, ester, or other physiologically functional derivative thereof and a pharmaceutically acceptable excipient whereby said cancerous condition is treated or prevented in said patient.

Yet another aspect of the present invention relates to a method of inhibiting or preventing the growth of a cancerous condition in a patient, comprising administering to the patient an effective amount of a compound of the formula

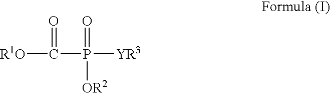

wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is a cytotoxic agent, or a pharmaceutically acceptable salt, ester, or other physiologically functional derivative thereof and a pharmaceutically acceptable excipient, whereby said growth of said cancerous condition is inhibited or prevented.

As used herein, the term “cancerous condition” relates to any condition where cells are in an abnormal state or condition that is characterized by rapid proliferation or neoplasia. A cancerous condition may be malignant or non-malignant (e.g. precancerous condition) in nature. Also used herein to further describe a “cancerous condition” are the terms “hyperproliferative”, “hyperplastic,” “hyperplasia,” “malignant”, and “neoplastic,” and “neoplasia.” These terms are used interchangeably and are meant to include all types of hyperproliferative growth, hyperplastic growth, cancerous growths or oncogenic processes, metastatic tissues or malignantly transformed cells, tissues or organs, irrespective of histopathologic type, stage of invasiveness, or cancerous determination (e.g. malignant and nonmalignant). As used herein, the term “neoplasia” refers to “new cell growth” that results in a loss of responsiveness to normal growth controls, e.g., neoplastic cell growth. A “hyperplasia” refers to cells undergoing an abnormally high rate of growth. However, as used herein, these terms can be used interchangeably, as their context will reveal, referring generally to cells experiencing abnormal cell growth rates. “Neoplasias” and “hyperplasias” include “tumors,” which may be either benign, premalignant, carcinoma in-situ, malignant, solid or non-solid. Examples of some cancerous conditions which are within the scope of the invention include, but are not limited to, anal cancer, breast cancer, ovarian cancer, cervical cancer, transitional cell bladder cancer, bronchogenic lung cancer, thyroid cancer, gastric cancer, head and neck cancer, ophthalmic cancers (e.g. retinoblastomas and other cancers of the eye), soft tissue sarcomas, osteogenic carcinomas (e.g. cancer of the bone), neuroblastomas, Wilms' tumor, malignant lymphoma, Hodgkin's lymphoma, renal cancers, Non-Hodgkin's lymphoma, leukemia, Kaposi's sarcoma, small cell lung cancer, and colorectal cancers. Other examples of non-malignant hyperproliferative conditions (e.g. precancerous conditions) that are within the scope of the invention include, but are not limited to, adenomas, chondromas, enchondromas, fibromas, myomas, myxomas, neurinomas, osteoblastomas, osteochondromas, osteomas, and papillary tumors.

As used herein, the terms “leukemia” or “leukemic cancer” refers to all cancers or neoplasias of the hematopoetic and immune systems (blood and lymphatic system). These terms refer to a progressive, malignant disease of the blood-forming organs, marked by distorted proliferation and development of leukocytes and their precursors in the blood and bone marrow. Myelomas refer to other types of tumors of the blood and bone marrow cells. Lymphomas refer to tumors of the lymph tissue. Examples of leukemia include acute myelogenous leukemia (AML), acute lymphoblastic leukemia (ALL), and chronic myelogenous leukemia (CML).

As used herein, the term “effective amount” refers to an amount of the chemotherapeutic agent of the present invention, which is effective, either alone or in combination with a pharmaceutical carrier, upon single- or multiple-dose administration to the subject, e.g., a patient, at inhibiting the growth or proliferation, inducing the killing, or preventing the growth of hyperproliferative cells. Such growth inhibition or killing can be reflected as a prolongation of the survival of the subject, e.g., a patient beyond that expected in the absence of such treatment, or any improvement in the prognosis of the subject relative to the absence of such treatment.

The term “treatment”, “treat” or “treated” refers to either (i) the prevention of tumor growth or regrowth of the tumor (prophylaxis), (ii) the reduction or elimination of symptoms or the disease of interest (therapy) or (iii) the elimination or destruction of the tumor (cure).

As used herein, “inhibiting the growth or proliferation” of the hyperproliferative cell, e.g. neoplastic cell, refers to the slowing, interrupting, arresting, or stopping its growth and metastasis, and does not necessarily indicate a total elimination of the neoplastic growth.

As used herein, the term “preventing” refers to the ability of the chemotherapeutic agent of the present invention to keep the growth or formation of a cancerous condition (e.g. neoplasia) from happening or existing. Also within the scope of the term “preventing” is the ability of the chemotherapeutic agent of the present invention or hold or keep back the growth or spread of an existing cancerous condition (e.g. neoplasia).

The term “patient” is intended to include human and nonhuman animals. Human animals include but are not limited to a human patient having a disorder characterized by the aberrant activity of a hyperproliferative cell. In one embodiment of the present invention, the patient will have at least one identifiable sign, symptom, or laboratory finding sufficient to make a diagnosis of a cancerous or precancerous condition in accordance with clinical standards known in the art for identifying such disorders. Examples of such clinical standards can be found in Harrison's Principles of Internal Medicine, 14th Ed., Fauci A S et al., eds., McGraw-Hill, New York, 1998. In some instances, a diagnosis of a cancerous condition will include identification of a particular aberrant (e.g. malignant or nonmalignant) cell type present in a sample of a body fluid or tissue obtained from the subject. The term “nonhuman animals” of the invention includes all vertebrates, e.g., mammals and non-mammals, such as nonhuman primates, sheep, dog, cat, horse, cow, chickens, amphibians, reptiles, and the like. In one embodiment, the subject is a human patient, e.g. a cancer patient.

The term “administering” or “administered” as used herein is meant to include both parenteral and/or oral administration. By “parenteral” is meant intravenous, subcutaneous or intramuscular administration. In the methods of the subject invention, the chemotherapeutic drug of the present invention may be administered alone, simultaneously with one or more other chemotherapeutic compounds, or the compounds may be administered sequentially, in either order. It will be appreciated that the actual method and order of administration will vary according to, inter alia, the particular preparation of chemotherapeutic agent being utilized, the particular formulation(s) of the one or more other chemotherapeutic compounds being utilized, the particular tumor cells being treated, and the particular host being treated. The method and order of administration of the compounds of the invention for a given set of conditions can be ascertained by those skilled in the art using conventional techniques and in view of the information set out herein. The term “administering” or “administered” also refers to oral sublingual, buccal, transnasal, transdermal, rectal, intramascular, intravenous, intraventricular, intrathecal, and subcutaneous routes. In accordance with good clinical practice, it is suggested that treating clinicians administer the instant compounds at a concentration level which will produce effective beneficial effects without causing any harmful or untoward side effects.

Another aspect of the invention relates to a method of treating or preventing a cancerous condition in a patient, comprising administering to the patient an effective amount of a compound of the formula

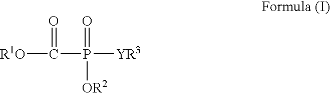

wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is a cytotoxic agent, or a pharmaceutically acceptable salt, ester, or other physiologically functional derivative thereof; and administering to said patient at least one additional chemotherapeutic drug, whereby said cancerous condition is treated or prevented.

The term “at least one additional chemotherapeutic drug” or “other chemotherapeutic agents” relates to those medications that are used to treat various forms of cancer, neoplasms, abnormal cell growth and the like. Generally, these medications are given in a particular regimen over a period of time. There are a number of different regimens available and their uses are dependent on a number of different factors, including, but not limited to, the type of cancerous condition being treated, the age and general health of the patient, and the stage of development of the cancerous condition. Examples of some chemotherapy regimens include, but are not limited to AVBD (Adriamycin, Vinblastine, Bleomycin and Dacarbizine), CHOP (Vincristine, Adriamycin, Prednisolone Cyclophosphamide), A.C.E. (Adriamycin, Cyclophosphamide, Etoposide), and F.A.C. (fluorouracil, Adriamycin, Cyclophosphamide). The optimal course of therapy for a given set of conditions can be ascertained by those skilled in the art using conventional course of therapy determination tests and in view of the information set out herein. It is an object of this invention that the chemotherapeutic agent of the present invention can be used as a part of, or as a replacement for one of the chemotherapeutic drugs in a given chemotherapeutic regimen.

The terms “induce,” “inhibit,” “potentiate,” “elevate,” “increase,” “decrease,” or the like denote quantitative differences between two states, and refer to at least statistically significant differences between the two states. For example, “an amount effective to inhibit the growth of hyperproliferative cells” means that the rate of growth of the cells will at least be statistically significantly different from the untreated cells. Such terms are applied herein to, for example, rates of proliferation.

Another aspect of the invention provides for a kit comprising a chemotherapeutic agent and a pharmaceutically acceptable excipient according to the present invention and instructions describing the use of the chemotherapeutic agent in treating a cancerous condition in a patient. In one embodiment, cancerous condition is a leukemia, e.g. acute myelogenous leukemia (AML), acute lymphoblastic leukemia (ALL) and chronic myelogenous leukemia (CML).

The present invention also provides for a method of treating or preventing a cancerous condition in a patient comprising administering to the patient an effective amount of a chemotherapeutic agent having one of the following structures:

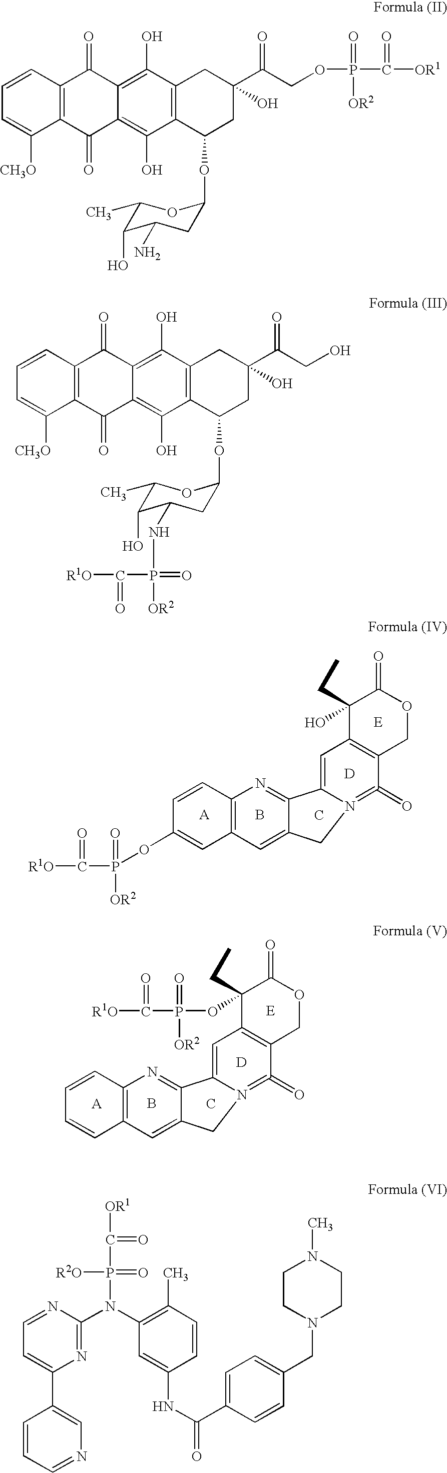

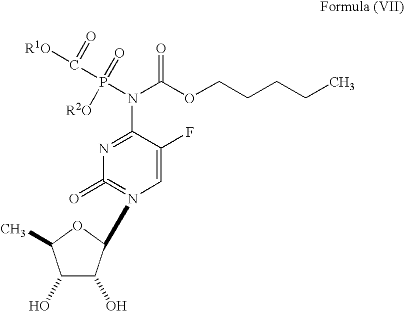

wherein R¹ is selected from the group consisting of methyl, alkyl, cholesteryl, aryl and aralkyl and R² is selected from the group consisting of hydrogen, methyl, alkyl, and a water-soluble cation, whereby the cancerous condition in the patient is treated or prevented. In one embodiment, the cancerous condition is selected from the group consisting of breast cancer, colorectal cancer, Adjuvant Stage III Dukes' colon cancer, acute myelogenous leukemia (AML), acute lymphoblastic leukemia (ALL) and chronic myelogenous leukemia (CML).

The present invention also provides for a method of inhibiting or preventing the growth of a cancerous cells in a patient comprising administering to the patient an effective amount of a chemotherapeutic agent having one of the following structures:

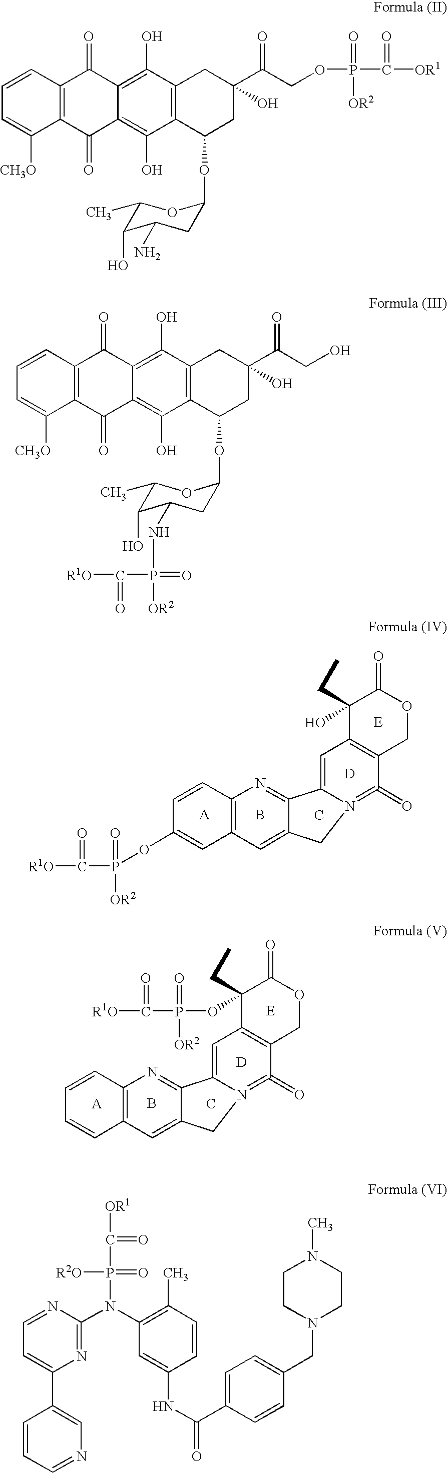

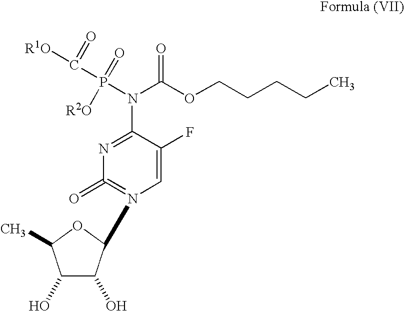

wherein R¹ is selected from the group consisting of methyl, alkyl, cholesteryl, aryl and aralkyl and R² is selected from the group consisting of hydrogen, methyl, alkyl, and a water-soluble cation, whereby the growth of the cancerous condition in the patient is inhibited or prevented. In one embodiment, the cancerous condition is selected from the group consisting of breast cancer, colorectal cancer, Adjuvant Stage III Dukes' C colon cancer, acute myelogenous leukemia (AML), acute lymphoblastic leukemia (ALL) and chronic myelogenous leukemia (CML).

It will be appreciated by those skilled in the art that compounds of the present invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-tumor activity using any tests which are well known in the art.

The description of the invention herein should be construed in congruity with the laws and principles of chemical bonding. An embodiment or aspect which depends from another embodiment or aspect, will describe only the variables having values and provisos that differ from the embodiment or aspect from which it depends. Thus, for example, an embodiment which reads “the compound of formula (I) according to the n^{th} aspect of the invention, wherein R¹ is CH₃” should be read to include all remaining variables with values defined in the nₜₕ aspect and should be read to further include all the provisos, unless otherwise indicated, pertaining to each and every variable in the n^{th} aspect. The numbers in the subscript after the symbol “C” define the number of carbon atoms a particular group can contain. For example “C₁₋₇alkyl” means a straight or branched saturated carbon chain having from one to seven carbon atoms, including without limitation groups such as methyl, ethyl, n-propyl, isopropyl, n-butyl, sec-butyl, isobutyl, t-butyl, n-pentyl, sec-pentyl, isopentyl, n-hexyl and n-heptyl. The term “halogen” includes fluoro, chloro, bromo and iodo. It is to be understood that the present invention includes any and all possible stereoisomers, geometric isomers, diastereoisomers, enantiomers, conformational isomers and anomers, unless a particular description specifies otherwise.

A chemotherapeutic agent of the present invention may be obtained as a single enantiomeric species by classical resolution with an enantiopure acid, such as mandelic acid, or by formation of readily separable diastereomers by an enantiopure derivatizing agent, or by chiral chromatography, or by enzymatic resolution of a compound of formula (I) or a suitable derivative, or by preparation of the compound of formula (I) from enantiopure precursors, which may themselves be obtained as single enantiomers by similar means.

The chemotherapeutic agents of the present invention, when used in pharmaceutical or diagnostic applications, may be in a purified form or not. Specifically, upon formation of the chemotherapeutic agent of the present invention, the mixture may be used immediately for preparation of a pharmaceutical composition or the desired salt may be purified. In one embodiment, the chemotherapeutic agent used in a pharmaceutical composition are prepared in substantially pure enantiomeric form, with an enantiopurity of at least 90% enantiomeric excess (EE), in one embodiment at least 95% EE, in another embodiment at least 98% EE, and in another embodiment at least 99% EE. Enantiomeric excess values provide a quantitative measure of the excess of the percentage amount of a major isomer over the percentage amount of a minor isomer which is present therewith, and may be readily determined by suitable methods well-known and established in the art, as for example chiral high pressure liquid chromatography (1-IPLC), chiral gas chromatography (GC), nuclear magnetic resonance (NMR) using chiral shift reagents, etc.

The present invention also contemplates pharmaceutical formulations, both, for veterinary and for human medical use, which comprise as the active agent one or more chemotherapeutic agent(s) of the present invention together with one or more pharmaceutically acceptable carrier(s) and optionally any other therapeutic ingredients, including one or more additional chemotherapeutic drugs, in the manufacture of a medicament for the treatment of the conditions and disorders variously described herein.

In such pharmaceutical and medicament formulations, the carrier(s) must be pharmaceutically acceptable in the sense of being compatible with the other ingredients of the formulation and not unduly deleterious to the recipient thereof. The chemotherapeutic agent (i.e. active agent) is provided in an amount effective to achieve the desired pharmacological effect, as described above, and in a quantity appropriate to achieve the desired daily dose.

The pharmaceutical compositions of the present invention may also comprise a pharmaceutically acceptable carrier excipient or diluent. Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-.beta.-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co, New Jersey (1991), incorporated herein by reference.

Pharmaceutical compositions of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof. Such pharmaceutical composition may be in the form of a freeze-dried mixture of the two active ingredients in a unit dosage form, prepared by conventional techniques, which can be reconstituted with water or other suitable infusion liquid at the time of administration.

The formulations also include those suitable for parental as well as nonparenteral administration; other specific administration modalities include intravenous, intraperitoneal, subcutaneous, rectal, topical, ophthalmic, subcutaneous, intrathecal, intra-articular, intra-arterial, subarachnoid, bronchial, lymphatic, intrauterine or intramuscular, all using dosage forms well known to those skilled in the pharmaceutical arts. The chemotherapeutic agents of the present invention can be administered in such oral dosage forms as tablets, capsules (each of which includes sustained release or timed release formulations), pills, powders, granules, elixirs, tinctures, suspensions, syrups and emulsions. Alternatively, the chemotherapeutic agents of the present invention may be administered bronchially, via nebulization of the powder in a carrier gas, to form a gaseous dispersion of the powder which is inspired by the patient from a breathing circuit comprising a suitable nebulizer device. The chemotherapeutic agents may also be administered alone but, as described above, generally will be administered with a pharmaceutical carrier selected on the basis of the chosen route of administration and standard pharmaceutical practice. Compounds of this invention can also be administered in intranasal form by topical use of suitable intranasal vehicles, or by transdermal routes, using transdermal skin patches. When compounds of this invention are administered transdermally the dosage will be continuous throughout the dosage regimen. Formulations suitable for parental administration are contemplated.

A tablet may be made by compression or molding, optionally with one or more accessory ingredients. Compressed tablets may be prepared by compressing in a suitable machine, with the chemotherapeutic agent of the present invention being in a free-flowing form such as a powder or granules which optionally is mixed with a binder, disintegrant, lubricant, inert diluent, surface active agent, or discharging agent. Molded tablets comprised of a mixture of the powdered active compound with suitable carrier may be made by molding in a suitable machine.

A syrup may be made by adding the chemotherapeutic agent of the present invention to a concentrated aqueous solution of sugar, for example sucrose, to which may be also be added any accessory ingredient(s). Such accessory ingredient(s) may include flavorings, suitable preservative(s), agents to retard crystallization of the sugar, and agents to increase the solubility of any other ingredient, such as polyhydroxy alcohol, for example glycerol or sorbitol.

Formulations suitable for parental administration conveniently comprise sterile aqueous preparation of the chemotherapeutic agent of the present invention, which in one embodiment is isotonic with the blood of the recipient (e.g., physiological saline solution). Such formulations may include suspending agents and thickening agents and liposomes or other microparticulate systems which are designed to target the chemotherapeutic agent to blood components or one or more organs. The formulations may be presented in unit-dose or multi-dose form.

Nasal spray formulations comprise purified aqueous solutions of the chemotherapeutic agents of the present invention with preservative agents and isotonic agents. Such formulations are in one embodiment adjusted to a pH and isotonic state compatible with the nasal mucous membranes.

Formulations for rectal administration may be presented as a suppository with a suitable carrier such as cocoa butter, hydrogenated fats, or hydrogenated fatty carboxylic acids.

Ophthalmic formulations are prepared by a similar method to the nasal spray, except that the pH and isotonic factors are in one embodiment adjusted to match that of the eye.

Topical formulations comprise the chemotherapeutic agent of the present invention dissolved or suspended in one or more media, such as mineral oil, petroleum, polyhydroxy alcohols, or other bases used for topical pharmaceutical formulations.

Transdermal formulations may be prepared by incorporating the chemotherapeutic agent of the present invention in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a patient wearer.

In general, while the effective dosage of the chemotherapeutic agents of the present invention are for achievement of a therapeutic benefit and depend in part on the specific application, condition, or disease state involved as well as the opinion of the treating physician. In one embodiment of the present invention, the effective dosage is in the range of 1 microgram ( [symbol] g) to 100 milligrams (mg) per kilogram body weight of the recipient per day, in another embodiment in the range of 5 [symbol] g to 75 mg per kilogram body weight per day and in another embodiment in the range of 10 [symbol] g to 50 mg per kilogram body weight per day. For example, in one embodiment, the chemotherapeutic agent comprises camptothecin or camptothecin analog covalently linked to a phosphonoformate ester. For parenteral administration of this composition, the course of therapy generally employed is from about 0.1 to about 300.0 mg/m² of body surface area per day for about one to about five consecutive days. In another embodiment, the course of therapy employed is from about 0.1 to about 100 mg/m² of body surface area per day for about five consecutive days. In another embodiment, the course of therapy employed for a chemotherapeutic agent of the present invention comprising topotecan is from about 1.0 to about 2.0 mg/m² of body surface area per day for about five consecutive days. In another embodiment, the course of therapy is repeated at least once at about a seven day to about a twenty-eight day interval (from the date of initiation of therapy) depending upon the initial dosing schedule and the patient's recovery of normal tissues. In another embodiment, the course of therapy continues to be repeated based on tumor response. In another embodiment, the parenteral administration of a chemotherapeutic agent of the present invention comprising camptothecin or camptothecin analog covalently linked to a phosphonoformate ester will be by short (e.g., 30 minute) or prolonged (e.g., 24 hour) intravenous infusion. In another embodiment, the compound will be administered by a 30 minute intravenous infusion. In any case, the dosage and dosage regimen and scheduling of a compound of the present invention must in each case be carefully adjusted, utilizing sound professional judgment and considering the age, weight and condition of the recipient, the route of administration and the nature and extent of the cancer disease condition.

Alternatively, orally administered dosages are typically at least twice, e.g. 2-10 times, the dosage levels used in parental administration methods, for the same active ingredient. For example, in oral administration for treating cancer, dosage levels for chemotherapeutic compounds of the present invention may be on the order of 5-200 mg/70 kg body weight/day. In one embodiment, the oral administration of a chemotherapeutic agent of the present invention comprising camptothecin or camptothecin analog covalently linked to a phosphonoformate ester, the course of therapy generally employed is from about 1.0 to about 500.0 mg/m² of body surface area per day for about one to five consecutive days. In another embodiment, the course of therapy employed is from about 1.5 to about 5.0 mg/m² of body surface area per day for about five consecutive days. In another embodiment, the course of therapy is repeated at least once at about a seven day to about a twenty-eight day interval (from the date of initiation of therapy) depending upon the initial dosing schedule and the patient's recovery of normal tissues. In another embodiment, the course of therapy continues to be repeated based on tumor response. In tablet dosage forms, typical active agent dose levels suitable for treating a cancerous condition are on the order of 10-100 mg per tablet.

Examples of pharmaceutically acceptable esters of the present invention include: (a) carboxylic acid esters of hydroxyl groups in compounds of formula (I) in which the non-carbonyl moiety of the carboxylic acid portion of the ester grouping is selected from straight or branched chain alkyl (e.g. n-propyl, t-butyl, n-butyl), alkoxyalkyl (e.g. methoxymethyl), arylalkyl (e.g. benzyl), aryloxyalky (e.g. phenoxymethyl), and aryl (e.g. phenyl); alkyl- or arylalkylsulfonyl (e.g. methanesulfonyl); amino acid esters (e.g. L-valyl or L-isoleucyl); dicarboxylic acid esters (e.g. dimethylaminocarbonyl, (2-aminoethyl)aminocarbonyl); and (b) alcohol esters of carboxylate groups in compounds of formula (I) in which the alcohol moiety of the ester grouping is selected from straight or branched chain alcohols (e.g. ethanol, t-butanol), phenols (e.g. 4-methoxyphenyl), alkoxyalcohols (e.g. ethoxyethanol), arylalkyl alcohols (e.g. benzyl alcohol), and aminoalcohols (e.g. 2-aminoethanol).

The chemotherapeutic agents of the present invention can also exist in the form of pharmaceutically acceptable salts. Such salts include addition salts with inorganic acids such as, for example, hydrochloric acid and sulfuric acid, and with organic acids such as, for example, acetic acid, citric acid, methanesulfonic acid, toluenesulfonic acid, tartaric acid and maleic acid. Further, in case the compounds of this invention that contain an acidic group, the acidic group can exist in the form of alkali metal salts such as, for example, a potassium salt and a sodium salt; alkaline earth metal salts such as, for example, a magnesium salt and a calcium salt; and salts with organic bases such as a triethylammonium salt and an arginine salt. The compounds of the present invention may be hydrated or non-hydrated.

In some applications, it may be advantageous to utilize the formula (I) composition of the present invention in a “vectorized” form, such as by encapsulation of the compound in a liposome or other encapsulant medium, or by fixation of the compound, e.g. by covalent bonding, chelation, or associative coordination, on a suitable biomolecule, such as those selected from proteins, lipoproteins, glycoproteins, and polysaccharides.

The formulations of the present invention may conveniently be presented in unit dosage forms and may be prepared by any of the methods well known in the art of pharmacy. Such methods generally include the step of bringing the active compound(s) into association with a carrier which constitutes one or more accessory ingredients. In one embodiment, the formulations are prepared by uniformly and intimately bringing the active compound(s) into associated with a liquid carrier, a finely divided solid carrier, or both, and then, if necessary, shaping the product into dosage forms of the desired formulation.

The one or more additional chemotherapeutic agents described here may be administered singly or in a cocktail containing both agents or one of the agents with other therapeutic agents, including but not limited to, immunosuppressive agents, potentiators and side-effect relieving agents

The pharmaceutical compositions of this invention which are found in combination may be in the dosage form of solid, semi-solid, or liquid such as, e.g., suspensions, aerosols or the like. In one embodiment the compositions are administered in unit dosage forms suitable for single administration of precise dosage amounts. The compositions may also include, depending on the formulation desired, pharmaceutically-acceptable, nontoxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solution, dextrose solution, and Hank's solution. In addition, the pharmaceutical composition or formulation may also include other carriers, adjuvants, or nontoxic, nontherapeutic, nonimmunogenic stabilizers and the like. Effective amounts of such diluent or carrier will be those amounts which are effective to obtain a pharmaceutically acceptable formulation in terms of solubility of components, or biological activity, and the like.

These and other aspects of the invention may become more readily apparent in connection with the following representative example which is presented for purposes of illustration and not by way of limitation.

### EXAMPLE

### Example 1

The compounds of the present invention can be prepared by the general procedure of reacting a trialkyl phosphonoformate with phosphorous pentachloride at room temperature or higher temperatures up to about 100° C., followed by the addition to the reaction mixture, at a low temperature, for example below 0° C., of the selected chemotherapeutic drug to form a triester having the structure shown in formulas I-VII above. The triester may then be selectively deesterified by reaction with sodium iodide at room temperature or higher in a suitable aprotic solvent, resulting in the desired compound in the form of the sodium salt. The sodium salt can readily be converted to other desired salts or to the free acid by conventional ion exchange procedures.

One example of preparation of the chemotherapeutic agents of the present invention may include adding phosphorous pentachloride to a solution of trimethyl phosphonoformate in carbon tetrachloride and warming the suspension to about 50° C. and stir for about 1.5 hours. The reaction mixture is evaporated to dryness under reduced pressure and the residue cooled to −50°. A solution of a desired chemotherapy drug in dry DMF, also pre-cooled to −50° C. is stirred and allowed to come to room temperature and concentrated to dryness under reduced pressure.

Another method of preparation may include stirring a desired chemotherapeutic agent into a solution of (ethoxycarbonyl)phosphonic dichloride in a solution of trimethyl phosphate at approximately 0° C. under a nitrogen atmosphere. After a few hours, the reaction mixture is concentrated to dryness with the aid of a vacuum pump. The residue is washed with ethanol, dried and taken up in formic acid. The formic acid is later removed by vacuum distillation.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

1. A chemotherapeutic agent having the structure 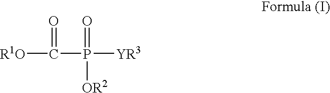
wherein R¹ is selected from the group consisting of methyl, alkyl, cholesteryl, aryl and aralkyl, R² is selected from the group consisting of hydrogen, methyl, alkyl, and a water-soluble cation, Y is selected from the group consisting of oxygen, sulfur, carbon and nitrogen, and R³ is anthracycline, a topoisomerase inhibitor, camptothecin, imatinib mesylate or capecitabine, wherein R³ is chemically bonded to Y.

2. The chemotherapeutic agent as claimed in claim 1, wherein said anthracycline is adriamycin.

3. A method for treatment of cancer for a patient in need thereof comprising, administering to the patient a therapeutically effective amount of a chemotherapeutic agent having the structure 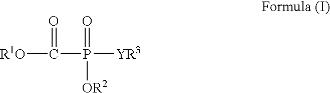
wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is a cytotoxic agent.

4. The method as claimed in claim 3, wherein the cytotoxic agent is an anthracycline.

5. The method of claim 4, wherein said anthracycline is adriamycin.

6. The method of claim 3, wherein the cytotoxic agent is a topoisomerase inhibitor.

7. The method of claim 6, wherein said topoisomerase inhibitor is camptothecin.

8. The method of claim 3, wherein the cytotoxic agent is imatinib mesylate.

9. The method of claim 3, wherein the cytotoxic agent is capecitabine.

10. The method of claim 3, wherein the patient has breast cancer, ovarian cancer, transitional cell bladder cancer, bronchogenic lung cancer, thyroid cancer, gastric cancer, soft tissue sarcomas, osteogenic carcinomas, neuroblastomas, Wilms' tumor, Adjuvant Stage III Dukes' C colon cancer, malignant lymphoma, Hodgkin's lymphoma, Non-Hodgkin's lymphoma, acute myelogenous leukemia, acute lymphoblastic leukemia, Kaposi's sarcoma, small cell lung cancer, colorectal cancers, or chronic myeloid leukemia.

11. The method of claim 3, wherein the patient has acute myelogenous leukemia.

12. The method of claim 3, wherein the patient has chronic myeloid leukemia.

13. The method of claim 3, wherein the patient has acute lymphoblastic leukemia.

14. The method of claim 3, wherein the patient has Adjuvant Stage III Dukes' C. colon cancer.

15. The method of claim 3, wherein the patient has breast cancer.

16. The method of claim 3, wherein the patient has colorectal cancer.

17. The method of claim 3, further comprising the step of administering to said patient an additional chemotherapeutic drug.

18. A method for treatment of cancer for a patient in need thereof comprising: administering to the patient a therapeutically effective amount of a chemotherapeutic agent having the structure 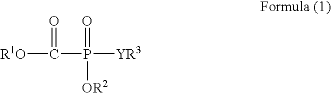
wherein R¹ is selected from the group consisting of methyl, alkyl, cholesteryl, aryl and aralkyl, Y is selected from the group consisting of oxygen, nitrogen, carbon and sulfur, R² is selected from the group consisting of hydrogen, methyl, alkyl, and a water-soluble cation, and R³ is selected from the group consisting of daunomycin, imatinib mesylate and adriamycin, wherein R³ is chemically bonded to Y.

19. The method of claim 18, wherein R³ is adriamycin and the patient has acute myeloblastic leukemia.

20. The method of claim 18, wherein R³ is adriamycin and the patient has acute lymphoblastic leukemia.
